(19) United States
(12) Patent Application Publication
(10) Pub. No.: US 2017/0032202 A1
(43) Pub. Date: Feb. 2, 2017

(54) Sweat Pores Imaging Method and Device

(71) Applicants: Uriel Halavee, Tel Aviv (IL); Arie Heiman, Sde Warburg (IL)

(72) Inventors: Uriel Halavee, Tel Aviv (IL); Arie Heiman, Sde Warburg (IL)

(21) Appl. No.: 15/212,236

(22) Filed: Jul. 17, 2016

Related U.S. Application Data
(60) Provisional application No. 62/231,885, filed on Jul. 20, 2015.

(51) Int. Cl. G06K 9/00; A61B 5/117; A61B 5/00

(52) U.S. Cl. CPC G06K 9/00885; A61B 5/4227; A61B 5/117; G06K 9/00087

(57) ABSTRACT

A biometric device, comprising: a sensor that reacts to a chemical element associated with a sweat gland; and when the sensor is contacted by an area of a skin of a person, an image that comprises visual information about locations of sweat glands within the area of the skin of the person

### CROSS REFERENCE TO RELATED APPLICATIONS

This application claims priority from US provisional patent filing date Jul. 17 2015, Ser. No. 62/231,885 which is incorporated herein by reference.

### BACKGROUND OF THE INVENTION

Fingerprint authentication by image acquisition and matching is one of the most widely used identification and verification method and is applied in field of criminal investigation, security, electronic transactions, banking etc.

Fingerprint scanners and imagers have become one of the most popular methods of identification in electronic devices and controlled access means in portable devices like smartphone and tablets; door locks, card readers, safes etc.

In general the authentication by fingerprint is based on two level criteria (algorithms) identifying fingerprint minutiae and pattern features like epidermal ridge flow map and frequency.

Some biometric systems combine both levels in the image processing protocol.

Despite the accepted uniqueness of fingerprints identification it is known that these methods are susceptible to forgery and counterfeit.

### SUMMARY

According to an embodiment of the invention there may be provided a biometric device that may include a sensor that may react to a chemical element associated with a sweat gland; and when the sensor may be contacted by an area of a skin of a person, an image that may include visual information about locations of sweat glands within the area of the skin of the person.

The sensor may be a sensor that may react with a chemical element included in sweat.

The sensor may be a moisture sensor that may include a hydrochromatic compound.

The image may include additional visual information about the area of the skin.

The additional visual information may include topographic information related to the area of the skin.

The additional visual information may include information about skin patterns.

The biometric device may include an authentication unit that may be configured to authenticate a person based on the visual information about the locations of the sweat glands and based on the additional visual information.

The moisture sensor may include a transparent polymer layer that may be doped with the hydrochromic compound.

The hydrochromic polymer layer may be deposited on a photoactive surface of the image sensor.

The hydrochromic polymer layer may be deposited on an optical component of the image sensor.

The hydrochromic layer may be deposited on a transparent polymer substrate and attached to the photoactive surface of the image sensor.

The biometric device where the hydrochromic layer may be deposited on a transparent polymer substrate doped with a light emitting phosphor and may be attached to a photoactive surface of the image sensor.

The skin biometric device where the hydrochromic layer may be deposited on an imaging light guide attached to the image sensor.

The moisture sensor may be transparent.

The biometric device may include an image processor that may be configured to process the image to generate a sweat glands map.

The biometric device may include an authentication unit that may be configured to process the image to authenticate a person.

The hydrochromic layer may be coated by a thin porous antifouling layer.

The biometric device may include a wireless communication module.

The field of view of the sensor may or may not exceed one square centimeter.

The field of view of the sensor exceeds one square centimeter.

According to an embodiment of the invention there may be provided a method for acquiring biometric information, the method may include acquiring, by an image sensor and when a sensor may be contacted by an area of a skin of a person, an image that may include visual information about locations of sweat glands within the area of the skin of the person; wherein the sensor may react to a chemical element associated with a sweat gland.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 illustrates biometric devices according to an embodiment of the invention;

FIG. 2 is an image of a finger brought in contact with a rectangular piece hydrochromic sheet and acquired using a smart phone camera according to an embodiment of the invention;

FIG. 3 is an image a skin segment of inner wrist brought in contact with a rectangular piece of hydrochromic sheet according to an embodiment of the invention;

FIG. 4 illustrates biometric devices according to an embodiment of the invention;

FIG. 5 illustrates biometric devices according to an embodiment of the invention; and

FIG. 6 illustrates a method according to an embodiment of the invention.

FIG. 7, presents a typical sweat gland images of two sample of the same finger that are overlaid one over the other according to an embodiment of the invention;

FIG. 8 presents a typical sweat gland images of two sample of two different fingers that are overlaid one over the other according to an embodiment of the invention; and

FIG. 9 illustrates a device according to an embodiment of the invention.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

According to an embodiment of the invention there may be provided a device that may include and a method that may use a sensor that may react to a chemical element associated with a sweat gland. For simplicity of explanation the following text will refer to a sensor that is a humidity sensor. It should be noted that the sensor may use chemo chromic detection of chemicals in the sweat such as lactate and others, or any combination of humidity sensing and any other chemical element appearing in the sweat.

The terms pores” and “glands” are used in an interchangeable manner.

Sweat glands are present on most of the human skin and distributed at varying concentration on different body regions. Their local density and exact location on the skin is unique and personal which makes a sweat gland map or print a powerful means of authentication.

There are provided methods and devices for acquisition of the sweat gland map on hydrochromic coating an image.

According to an embodiment of the invention there may be provided a low cost skin biometric device for imaging and mapping of sweat glands and registration of sweat glands distribution on skin for cosmetic, medical and authentication applications and specifically for combined imaging of sweat glands together with other skin identification marks for improved identification and verification.

The suggested device and methods provide a higher level of secure verification in comparison to prior art methods and devices—as the suggested method and devices provide an additional level of verification based on acquisition of a fingerprint based on moisture distribution which displays a map of sweat glands together with the standard fingerprint detection process. Such a map is much more difficult to forge than fingerprints.

In combination with fingerprint acquisition obtained by bringing the finger in contact with the surface of a moisture permeable polymer doped with a hydrochromic compound an image is generated which contains all three levels of authentication.

In an embodiment the current invention presents a device which comprises a image sensor such as a CCD or CMOS combined with a transparent polymer layer doped with a dispersion of reversible hydrochromic, colorimetric moisture indicating composition optimized for fast response both in acquisition and recovery (fast change in color and fast return to original state) for repeatable operation.

Many hydrochromic materials are known in the art and such materials can be used in this invention. They may consist of various salts as well as polymer dyes. Preferably, the hydrochromic material comprises cobalt chloride or copper chloride, cobalt chloride being the preferred. These hydrochromic materials can be incorporated in hydrophilic matrices like PVA, polyacrylamide, PEG etc. to produce hydrochromic sheets and layers.

The change of color on exposure to moisture can be irreversible for single use or reversible after evaporation.

The device when brought in contact with a skin area records the change in the moisture sensitive hydrochromic layer which corresponds to local moisture distribution and enables the simultaneous acquisition of the position of many sweat glands together with the local skin features, fingerprint ridge map, wrinkle map or local skin identification marks.

In one embodiment the hydrochromic polymer layer is deposited directly on the photosensitive surface of a CCD or CMOS image sensor by coating, adhesion, spraying, spin coating and other deposition methods known in the art.

In another embodiment the hydrochromic polymer layer is deposited on a transparent solid substrate mounted in direct contact with the image sensor photosensitive surface.

In another embodiment the solid substrate contains a light emitting phosphor or tiny waveguide or fiber optic which supplies illumination.

In some embodiments, the colorimetric hydrochromic layer of the device is overlaid by a thin transparent, porous, water permeable self cleaning layer for prevention of fouling or any erasable layer.

In a further embodiment the device comprises a hydrochromic polymer layer deposited on a transparent substrate (glass, polymer or other) and mounted together with a photo camera, web camera, smart phone camera and enabled to acquire a sweat gland map image together with the skin features.

In a further embodiment the device is a standalone device enabled with wireless connectivity like Wi Fi, Bluetooth or NFC enabling the transfer of the image for image processing and analysis.

In a further embodiment the biometric device of invention is equipped with a light source and power source.

In a further embodiment the device is enabled with wireless charging.

The device of invention can be incorporated in any electronic device requiring authentication: computer, Smartphone, smart watch, teller machine, cash register, access control systems, lock etc.

In further embodiment the hydrochromic layer is deposited on an imaging light guide or fiber bundle coupled to the image sensor.

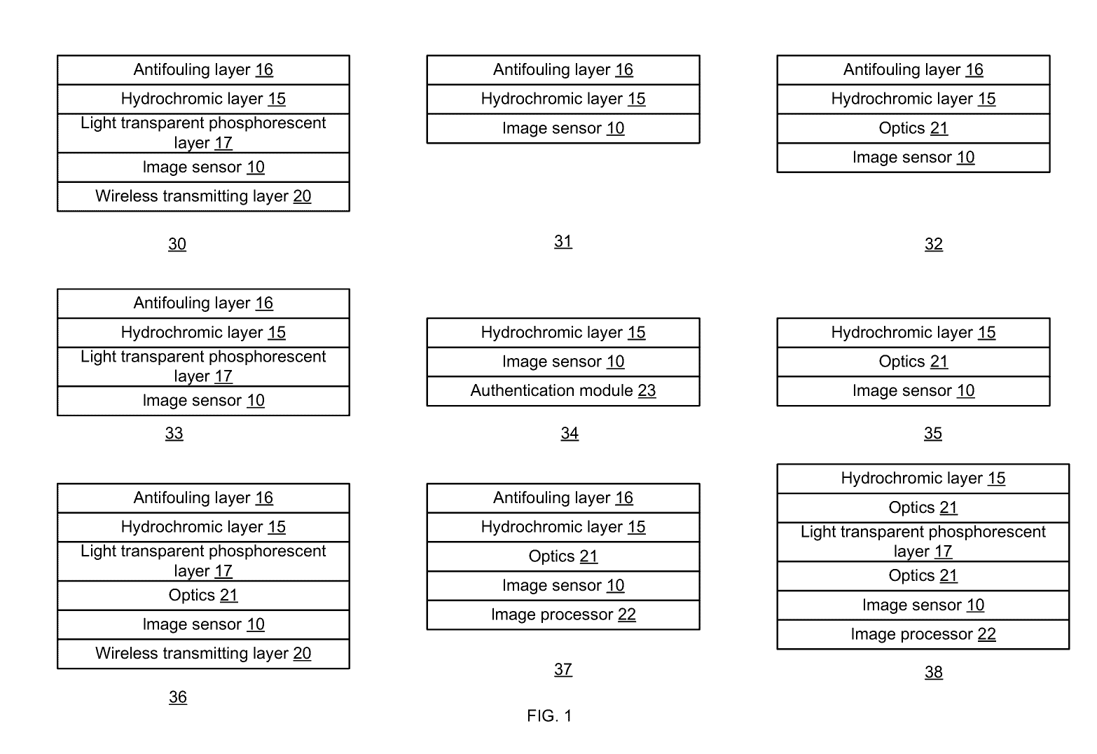

FIGS. 1, 4 and 5 illustrate various biometric devices 30, 31, 32, 33, 34, 35, 36, 37, 38, 50, 51, 52, 53, 54, 55, 56, 57, 58, 60, 61 and 62 according to various embodiment of the invention.

Biometric device 30 includes the following sequence of elements: antifouling layer 16, hydrochromic layer 15, light transparent phosphorescent layer 17, image sensor 10 and wireless transmitting layer 20. The hydrochromatic layer 15 is a non-limiting example of a moisture sensor that includes a hydrochromatic compound. The antifouling can be also done by erasable layer.

Biometric device 31 includes the following sequence of elements: antifouling layer 16, hydrochromic layer 15 and image sensor 10.

Biometric device 32 includes the following sequence of elements: antifouling layer 16, hydrochromic layer 15, optics 21 and image sensor 10. Optics 21 may be any optical lens or optical element for conveying, relaying, filtering, magnifying, manipulating radiation. The optics may include an optical guide, one or more lenses, spatial and/or spectral filters, polarizing elements and the like.

Biometric device 33 includes the following sequence of elements: antifouling layer 16, hydrochromic layer 15 and image sensor 10.

Biometric device 34 includes the following sequence of elements: hydrochromic layer 15, image sensor 10 and authentication module 23.

Biometric device 35 includes the following sequence of elements: hydrochromic layer 15, optics 21 and image sensor 10.

Biometric device 36 includes the following sequence of elements: antifouling layer 16, hydrochromic layer 15, light transparent phosphorescent layer 17, optics 21, image sensor 10 and wireless transmitting layer 20.

Biometric device 37 includes the following sequence of elements: antifouling layer 16, hydrochromic layer 15, optics 21, image sensor 10 and image processor 22.

Biometric device 38 includes the following sequence of elements: hydrochromic layer 15, optics 21, light transparent phosphorescent layer 17, optics 21, image sensor 10 and image processor 22.

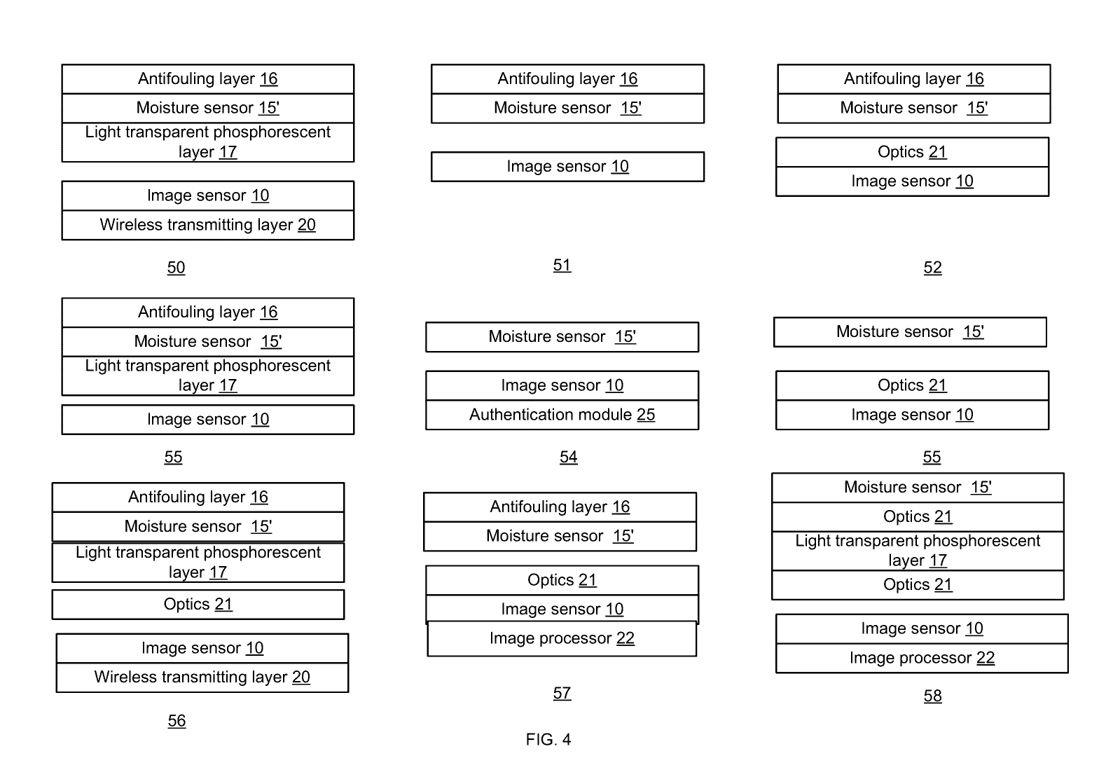

Biometric devices 50, 51, 52, 53, 54, 55, 56, 57 and 58 of FIG. 4 differ from biometric devices 30-38 of FIG. 1 by (a) having a gap between image sensor 10 and/or optics 21 and one or more other parts of the biometric device, and by (b) having a moisture sensor 15′ instead of hydrochromatic layer 15. The moisture sensor that includes a hydrochromatic compound.

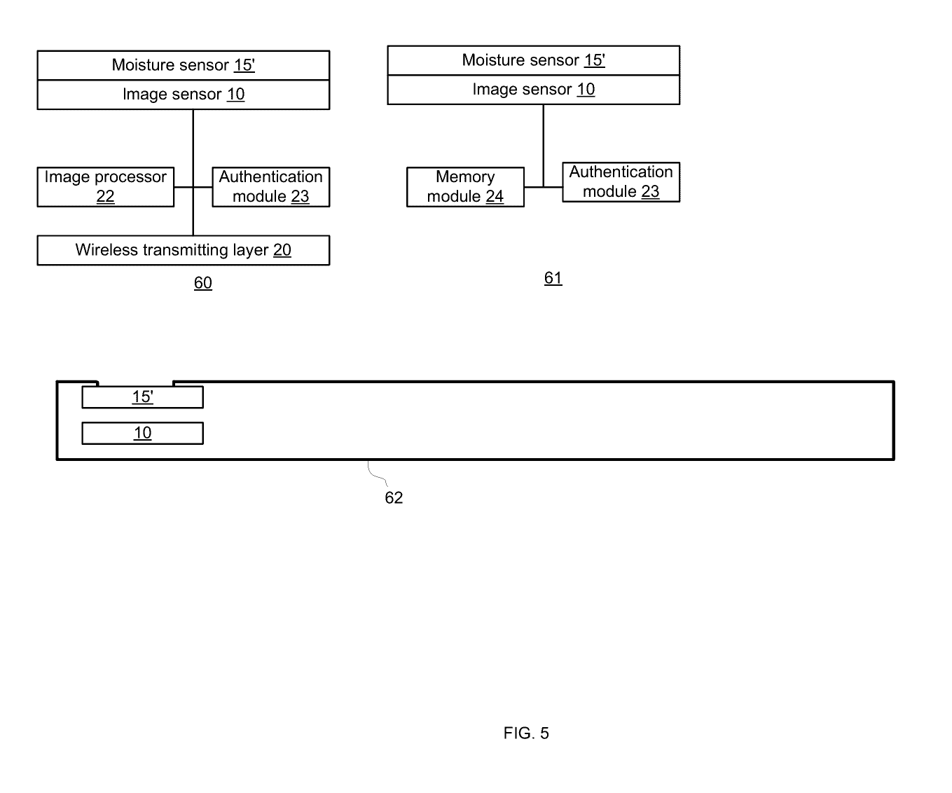

FIG. 5 biometric devices 60 and 61 as well as a cross section of a device 62 (such as but not limited to a smartphone) that includes moisture sensor 15′ and image sensor 10. The device 62 may include any combination of any of biometric devices 30-38, 59-60.

Biometric device 60 includes moisture sensor 15′, image sensor 10, wireless transmitting layer 20, image processor 22 and authentication module 23.

Biometric device 61 includes moisture sensor 15′, image sensor 10, wireless transmitting layer 20, image processor 22 and authentication module 23.

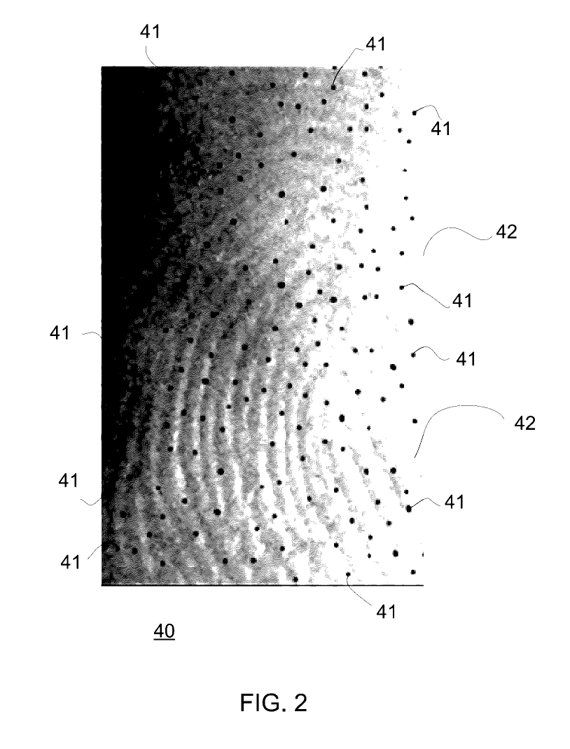

FIG. 2 is an image of a finger brought in contact with a rectangular piece hydrochromic sheet and acquired using a smart phone camera according to an embodiment of the invention. The image includes topographic information 42 as well as information about the location of the sweat glands—such as dots 41.

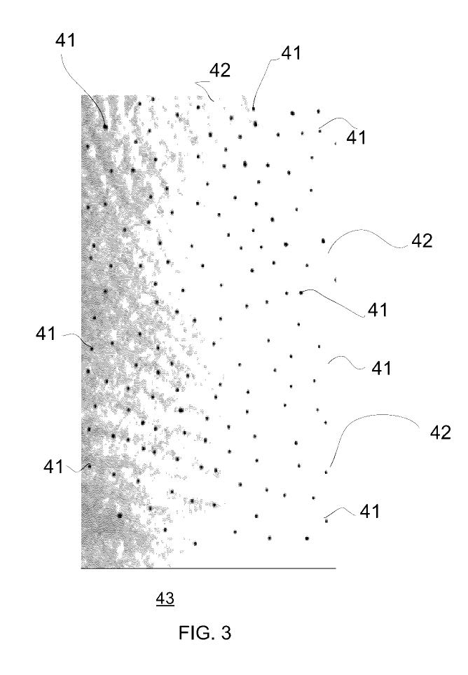

FIG. 3 is an image a skin segment of inner wrist brought in contact with a rectangular piece of hydrochromic sheet according to an embodiment of the invention. The image includes topographic information 42 as well as information about the location of the sweat glands—such as dots 41.

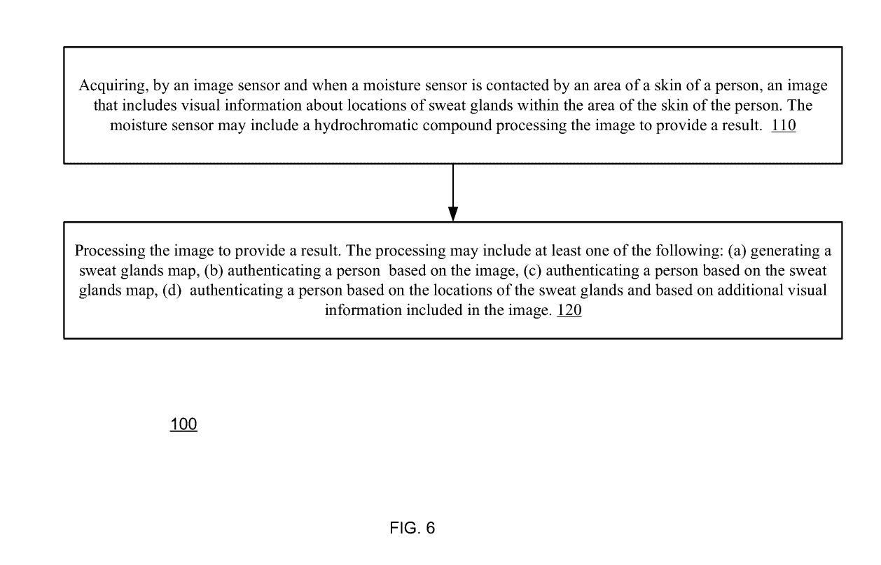

FIG. 6 illustrates method 100 according to an embodiment of the invention.

Method 100 may start by step 110 of acquiring, by an image sensor and when a moisture sensor is contacted by an area of a skin of a person, an image that includes visual information about locations of sweat glands within the area of the skin of the person. The moisture sensor may include a hydrochromatic compound.

As indicated above- the image may be acquired when a sensor (even a sensor that differs from a moisture sensor) reacts to a chemical element associated with a sweat gland.

The area may be large enough to acquire enough information that can be used to authenticate a person. For example—if an area of skin of a given size (for example a square centimeter) is large enough to include enough sweat gland information to identify a person then the area of the skin should be of that size or may be bigger than that size. Alternatively—the person may scan more than a single area of skin to provide enough information for authentication.

The moisture sensor may include a hydrochromatic layer or any other shaped arrangement of the hydrochromatic compound.

The moisture sensor may be transparent in order to enable an image sensor to acquire the image while the area of the skin contacts the image sensor.

The image acquired during step 110 may also include additional visual information about the area of the skin.

The additional visual information may include includes topographic information related to the area of the skin. The topographic information refers to the three dimensional shape of the area of the skin. For example- if the area of the skin is a part of a finger than the topographic information may include a part of the fingerprint.

The additional visual information may include information about skin patterns.

Step 110 may be followed by step 120 of processing the image to provide a result.

The processing may include at least one of the following: (a) generating a sweat glands map, (b) authenticating a person based on the image, (c) authenticating a person based on the sweat glands map, (d) authenticating a person based on the locations of the sweat glands and based on additional visual information included in the image.

The authentication process may include comparing the information in the image to reference information such as a reference sweat glands map and/or reference fingerprint information (or other visual information such as topographic information) of a person.

Step 120 may be executed by the biometric device or by another device. The other device may receive the image by wireless transmission or any other manner.

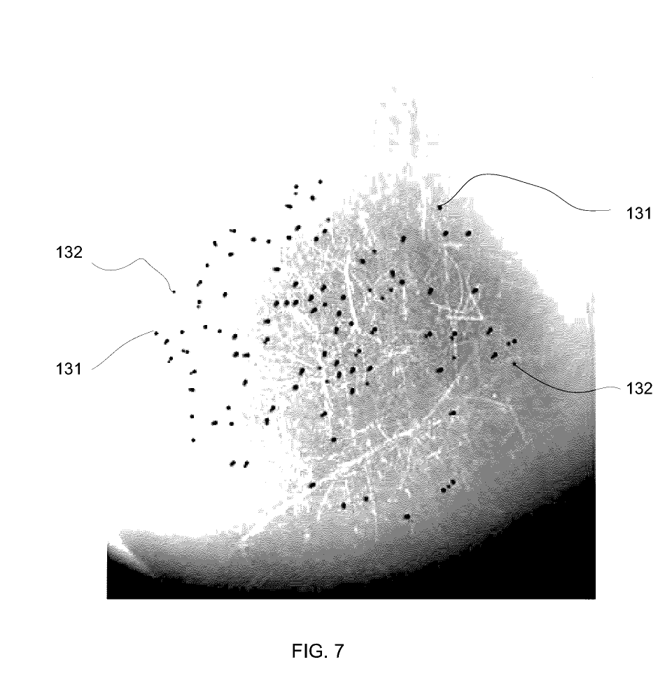

FIG. 7, presents a typical sweat gland images of two sample of the same finger that are overlaid one over the other. The two images (maps) are very similar to each other. Dots 131 provide an indication about the location of the sweat glands of the first sample. Dots 132 provide an indication about the location of the sweat glands of the second sample.

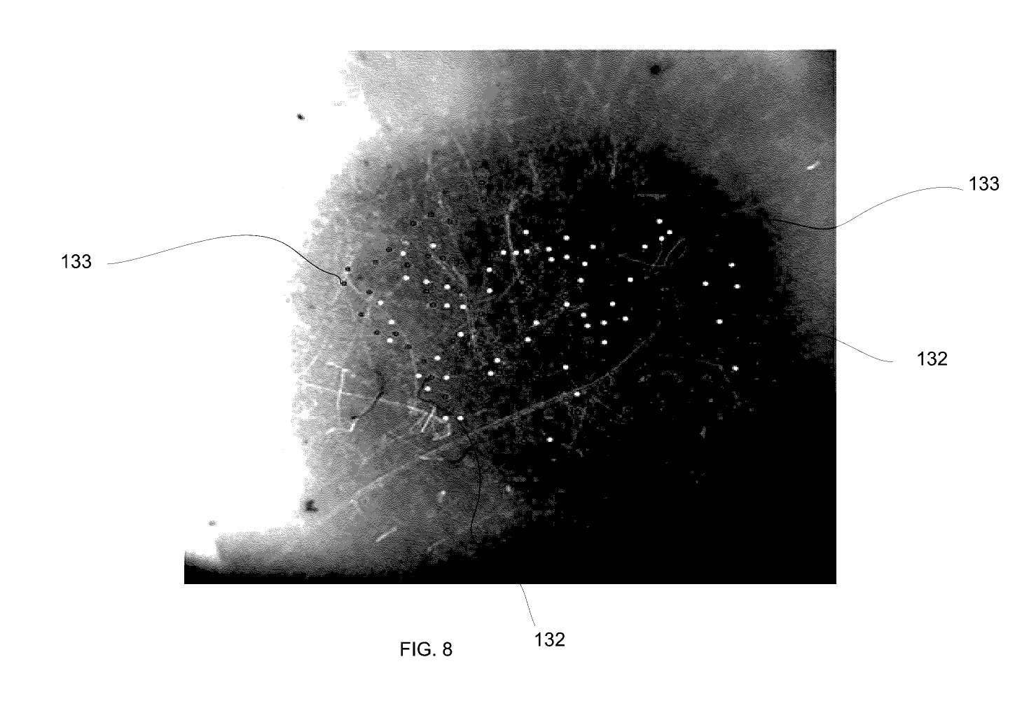

FIG. 8 presents a typical sweat gland images of two sample of two different fingers that are overlaid one over the other. The two images (maps) are totally different.

Dots 131 provide an indication about the location of the sweat glands of the first sample. Dots 133 provide an indication about the location of the sweat glands of the second sample (oft he other finger).

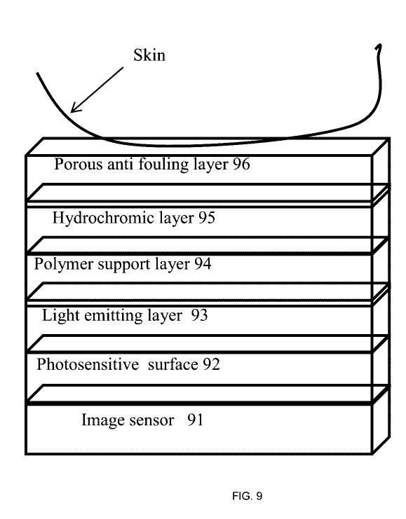

In FIG. 9 elements 91 and 92 represents the image sensor with its photosensitive surface. 93 is a transparent light source such as phosphor or tiny waveguide or fiber optic waveguide this layer can be located in different location. Layer 94 is a polymer that support the upper layers. 95 is Hydrochromic layer and or chemo chromic layer that are sensitive to the sweat, 96 is Porous anti fouling layer. It is noted that one or more of the elements of FIG. 9 may be merged to a single layer.

In the foregoing specification, the invention has been described with reference to specific examples of embodiments of the invention. It will, however, be evident that various modifications and changes may be made therein without departing from the broader spirit and scope of the invention as set forth in the appended claims.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

The connections as discussed herein may be any type of connection suitable to transfer signals from or to the respective nodes, units or devices, for example via intermediate devices. Accordingly, unless implied or stated otherwise, the connections may for example be direct connections or indirect connections. The connections may be illustrated or described in reference to being a single connection, a plurality of connections, unidirectional connections, or bidirectional connections. However, different embodiments may vary the implementation of the connections. For example, separate unidirectional connections may be used rather than bidirectional connections and vice versa. Also, plurality of connections may be replaced with a single connection that transfers multiple signals serially or in a time multiplexed manner. Likewise, single connections carrying multiple signals may be separated out into various different connections carrying subsets of these signals. Therefore, many options exist for transferring signals.

Although specific conductivity types or polarity of potentials have been described in the examples, it will be appreciated that conductivity types and polarities of potentials may be reversed.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements.

The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

We claim:

1. A biometric device, comprising: a sensor that reacts to a chemical element associated with a sweat gland; and when the sensor is contacted by an area of a skin of a person, an image that comprises visual information about locations of sweat glands within the area of the skin of the person.

2. The biometric device according to claim 1 wherein the sensor is a sensor that reacts with a chemical element included in sweat.

3. The biometric device according to claim 1 wherein the sensor is a moisture sensor that comprises a hydrochromatic compound.

4. The biometric device according to claim 1, wherein the image further comprises additional visual information about the area of the skin.

5. The biometric device according to claim 4, wherein the additional visual information includes topographic information related to the area of the skin.

6. The biometric device according to claim 4, wherein the additional visual information includes information about skin patterns.

7. The biometric device according to claim 4 , comprising an authentication unit that is configured to authenticate a person based on the visual information about the locations of the sweat glands and based on the additional visual information.

8. The biometric device according to claim 3, wherein the moisture sensor comprises a transparent polymer layer that is doped with the hydrochromic compound.

9. The biometric device according to claim 8, wherein the hydrochromic polymer layer is deposited on a photoactive surface of the image sensor.

10. The biometric device according to claim 9, wherein the hydrochromic polymer layer is deposited on an optical component of the image sensor.

11. The biometric device according to claim 9, wherein the hydrochromic layer is deposited on a transparent polymer substrate and attached to the photoactive surface of the image sensor.

12. The biometric device according to claim 9, where the hydrochromic layer is deposited on a transparent polymer substrate doped with a light emitting phosphor and is attached to a photoactive surface of the image sensor.

13. The skin biometric device according to claim 9, where the hydrochromic layer is deposited on an imaging light guide attached to the image sensor.

14. The biometric device according to claim 3, wherein the moisture sensor is transparent.

15. The biometric device according to claim 1, further comprising an image processor that is configured to process the image to generate a sweat glands map.

16. The biometric device according to claim 1, further comprising an authentication unit that is configured to process the image to authenticate a person.

17. The biometric device according to claim 3, wherein the hydrochromic layer is coated by a thin porous antifouling layer.

18. The biometric device according to claim 1, comprising a wireless communication module.

19. The biometric device according to claim 1, wherein a field of view of the sensor does not exceed one square centimeter.

20. The biometric device according to claim 1, wherein a field of view of the sensor exceeds one square centimeter.

21. A method for acquiring biometric information, the method comprises acquiring, by an image sensor and when a sensor is contacted by an area of a skin of a person, an image that comprises visual information about locations of sweat glands within the area of the skin of the person; wherein the sensor reacts to a chemical element associated with a sweat gland.

22. The method according to claim 21 wherein the sensor is a moisture sensor that comprises a hydrochromatic compound.

23. The method according to claim 21, comprising processing the image to generate a sweat glands map.
